(19) United States
(12) Patent Application Publication
(10) Pub. No.: US 2004/0186158 A1
(43) Pub. Date: Sep. 23, 2004

(54) Novel composition of carvedilol

(76) Inventor: Choon K. Oh, Collegeville, PA (US)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/491,195

(22) PCT Filed: Oct. 1, 2002
(86) PCT No.: PCT/US02/31298

(30) Foreign Application Priority Data
Oct. 2, 2001 (US) 60326616

(51) Int. Cl. A61K 31/403

(52) U.S. Cl. 514/411

(57) ABSTRACT

This invention relates to a novel composition of carvedilol and the use of such a composition in treatment of pediatric heart failure.

### FIELD OF THE INVENTION

This invention relates to a novel composition of carvedilol and to the use of such a composition in treatment of pediatric heart failure.

### BACKGROUND OF THE INVENTION

The compound, 1-(carbazol-4-yloxy-3-[[2-(o-methoxyphenoxy)ethyl]amino]-2-propanol, is known by the name “carvedilol” and is the subject of U.S. Pat. No. 4,503,067 (the '067 patent), issued Mar. 5, 1985. This compound has the following structure:

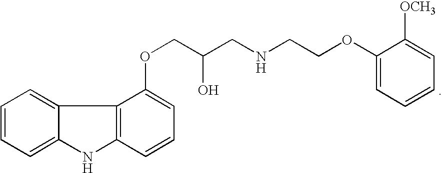

Carvedilol is useful in the treatment of hypertension, congestive heart failure and angina.

The current commercial formulation for carvedilol is immediate release, and it is administered twice daily in the adult patient population. However, there is a need to develop a novel composition for use in treatment of heart failure in the pediatric population.

According to the instant invention, it has been found that carvedilol can be formulated in a novel composition for use in treatment of pediatric heart failure.

### SUMMARY OF THE INVENTION

The present invention provides for the use of a composition comprising carvedilol in the treatment of pediatric heart failure.

### DESCRIPTION OF THE INVENTION

According to the present invention, a composition of carvedilol is provided for as an oral suspension for use in the treatment of pediatric heart failure. Carvedilol tablets (3.125 mg, 6.25 mg, 12.5 mg, and 25 mg) are supplied to the clinic in bottles. Pharmacists in the clinic prepare the oral suspensions by disintegrating a tablet in a small amount of water in the dispensing bottle and compounding in a mixture of 15 mL Ora-Plus® oral suspending vehicle and 10 mL Ora-Sweet® syrup vehicle which are commercially available. Based upon body weight, the appropriate amount of suspension is delivered utilizing a plastic syringe.

No unacceptable toxicological effects are expected when carvedilol is administered in accordance with the present invention.

The following examples are illustrative of the instant invention. These examples are not intended to limit the scope of this invention as defined hereinabove and as claimed hereinbelow.

### EXAMPLES

I. Components and Composition of the Drug Product

The tablets contain carvedilol equivalent to 3.125 mg, 6.25 mg, 12.5 mg and 25 mg. The unit formula for the tablets is presented below.

Unit Formula

TABLE 1
Unit Formulae for Carvedilol Tablets
(3.125 mg, 6.25 mg, 12.5 mg & 25 mg)

| | Strength | | | |
|---|---|---|---|---|
| | 3.125 mg | 6.25 mg | 12.5 mg | 25 mg |
| Ingredients | Quantity (mg/tablet) | | | |
| SPRAY SUSPENSION | | | | |
| Carvedilol | 3.125 | 6.25 | 12.5 | 25 |
| Lactose, NF (Impalpable) | 1.25 | 2.50 | 5.0 | 10 |
| Sucrose, NF (Extra Fine | 0.625 | 1.25 | 2.5 | 5 |
| Granulated) | | | | |
| Povidone, USP (K 29-32) | 0.125 | 0.25 | 0.5 | 1 |
| Colloidal silicon dioxide, NF | 0.25 | 0.50 | 1.0 | 2 |
| Purified water, USP | qs | qs | qs | qs |
| BULKING AGENTS | | | | |
| Crospovidone, NF | 1.875 | 3.75 | 7.5 | 15 |
| Lactose, NF (Fast-Flo ™) | 27.625 | 55.25 | 110.5 | 221 |
| EXTERNALS | | | | |
| Crospovidone, NF | 1.875 | 3.75 | 7.5 | 15 |
| Colloidal silicon dioxide, NF | 0.375 | 0.75 | 1.5 | 3 |
| Magnesium stearate, NF | 0.375 | 0.75 | 1.5 | 3 |
| TOTAL CORE WEIGHT | 37.5 | 75.0 | 150.0 | 300 |
| COATING | | | | |
| White Opadry YS-1-7003 | 1.125 | 2.25 | 4.5 | 9 |
| Purified water, USP | qs | qs | qs | qs |
| Clear Opadry YS-2-7013 | 0.15 | 0.30 | 0.6 | 1.2 |
| Purified water | qs | qs | qs | qs |
| TOTAL AFC TABLET | 38.775 | 77.55 | 155.1 | 310.2 |
| WEIGHT | | | | |

II. Method of Manufacturing the Drug Product

Manufacturing Process

The following process summary is for the 3.125, 6.25, 12.5 and 25 mg tablet strengths. The four tablet strengths are compressed from a common granulation and have the same coating procedure.

1. Suspension preparation

1.1 Weigh the Sucrose, NF (extra fine granulated), Lactose, NF (impalpable), Povidone, USP (K29-32), Colloidal silicon dioxide, NF and carvedilol. Weigh the Lactose, NF (Fast-Flo™) and Crospovidone, NF after it has been previously sieved with a Glatt Quick Sieve or equivalent equipment.

1.2 Charge the Sucrose, NF (extra fine granulated), the Lactose, NF (impalpable) and Povidone, USP (K29-32) in the Purified water, USP, previously heated at 50±5° C. Use a homogenizer or an air mixer to assist in dissolving the materials.

1.3 Disperse the Colloidal silicon dioxide, NF in the solution of Step 1.1.

1.4 Suspend the carvedilol in the slurry of Step 1.3 by adding it gradually while agitating and mix.

1.5 Pass the suspension through a Sylverson High Shear homogenizer or equivalent equipment.

2. Granulation

2.1 Charge Lactose, NF (Fast-Flo™) and Crospovidone, NF into a fluid bed drier.

2.2 Granulate the Lactose, NF and Crospovidone, NF in the fluid bed drier by spraying in the suspension of Step 1.5.

2.3 Dry the granulation to an L.O.D. of 1.7%±0.4.

2.4 Weigh the dried carvedilol granulation and screen the dried granulation through a Glatt Quick Sieve or equivalent equipment.

3. Lubrication and tableting

3.1 Transfer to a bin tumble blender.

3.2 Weigh, screen and charge the Crospovidone, NF, Colloidal silicon dioxide, NF, and Magnesium stearate, NF to the bin tumble blender.

3.3 Mix the powders.

3.4 Using the appropriate tooling, compress the final mix on a rotary tablet press.

4. Coating

4.1 Film coat the tablets in a perforated coating pan by spraying them with an aqueous dispersion of Opadry White YS-1-7003 followed by a finishing coat of an aqueous solution of Opadry Clear YS-2-7013.

It is to be understood that the invention is not limited to the embodiments illustrated hereinabove and the right is reserved to the illustrated embodiments and all modifications coming within the scope of the following claims. The various references to journals, patents and other publications which are cited herein comprise the state of the art and are incorporated herein by reference as though fully set forth.

What is claimed is:

1. A composition comprising carvedilol as an oral suspension.

2. The composition according to claim 1 which further comprises an oral suspending agent vehicle and a syrup vehicle.

3. The composition according to claim 2 wherein the oral suspending agent vehicle is Ora-Plus® suspending agent vehicle.

4. The composition according to claim 2 wherein the syrup vehicle is Ora-Sweet® syrup vehicle.

5. A method of treating pediatric heart failure which comprises administering to a subject in need thereof an effective amount of the composition according to any one of claims 1-4.

6. The use of the composition according to claims 1-4 in the manufacture of a medicament for the treatment of pediatric heart failure.
